(19) United States
(12) Patent Application Publication
(10) Pub. No.: US 2008/0014115 A1
(43) Pub. Date: Jan. 17, 2008

(54) Blood/Air Mass Exchange Apparatus

(75) Inventor: William Richard Johns, Reading (GB)

Correspondence Address: MG-IP Law, PLLC, P.O. BOX 1364, FAIRFAX, VA, 22038-1364, US

(73) Assignee: HAEMAIR LIMITED, SWANSEA, UNITED KINGDOM (GB)

(21) Appl. No.: 11/628,312

(22) PCT Filed: May 27, 2005
(86) PCT No.: PCT/GB05/02113

§ 371 Date: Dec. 4, 2006

(30) Foreign Application Priority Data
Jun. 3, 2004 (GB) 0412315.4

(51) Int. Cl. A61M 1/16

(52) U.S. Cl. 422/46; 422/45

(57) ABSTRACT

There is provided a mass exchange apparatus for use in blood/air mass exchange comprising plural blood flow conduits for defining blood flow; and plural air flow conduits for defining air flow. The plural air flow conduits and the plural blood flow conduits at least partially comprise gas-permeate membrane material, and the conduits are arranged relative to each other such as to enable transfer of oxygen from the air to the blood and transfer of carbon dioxide from the blood to the air. The blood and air do not directly come into contact (i.e. the mass exchange is indirect). There is also provided a prosthetic lung comprising an elastic bellows and the at least one mass exchange apparatus herein. There is further provided an external respiratory aid to augment patient lung function comprising the at least one mass exchange apparatus herein and means to pump air and blood through the apparatus. There is further provided an intermediate respiratory aid apparatus for internal connection to a patient comprising at least one mass exchange apparatus herein and an air pump.

### TECHNICAL FIELD

The present invention relates to a compact blood/air mass exchange apparatus for use in either a prosthetic lung suitable for use internally within the body of the patient (i.e. as a ‘prosthetic lung’) or with an external or part-external respiratory aid.

### BACKGROUND TO THE INVENTION

In Europe and North America, there are currently about 10,000 people on lung-transplant waiting lists. Each year, about 2500 people are transplanted, of whom approximately 2000 survive to live healthy lives. Each year about 2500 die on the waiting list, during a typical 2-year waiting period. The situation is actually far worse than the statistics would indicate because a much larger number of people are never entered onto waiting lists. These people may be excluded because they have no chance of surviving the wait for a transplant or because they are too old. There is little prospect that the situation will improve because the availability of donor organs is declining.

The controversial solution of xeno-transplantation appears to remain in the distant future. The availability of suitable prosthetic lungs would revolutionize the situation. The clinical trials requirements are likely to be more straightforward for prosthetics than for xeno-transplantation, and consequently, the potential time scale for introduction of prosthetic lungs is likely to be shorter. To date, the development of prosthetic lungs has been deterred because of the perceived difficulty involved in reproducing the structure and function of a human lung.

It is known that human lungs have a complex system of branching tubes leading to a multiplicity of small air sacs in which counter-diffusion (oxygen with carbon dioxide) takes place. The Applicant has realized that the engineering challenge in reproducing this kind of structure precludes any prosthesis that directly mimics the human lung.

The Applicant has now developed a prosthetic lung having a structure that is simpler than that of a human lung, but capable of comparable respiratory function. Such structure is both amenable to incorporation into a prosthetic lung for ‘transplant’ into the body of a patient and in an alternative use, as part of an external or intermediate respiratory aid. Applicant's solution comprises a mass exchange apparatus that functions as a counter-diffusion device to transfer oxygen from the air into the blood and carbon dioxide from the blood to the air. The blood and air flow in alternate channels or conduits. The walls defining the channels or conduits are gas-permeable to allow the required mass transfer. The conduits or channels could be defined by a series of plates that are separated by a small distance (e.g. a fraction of a millimetre). Alternatively, the conduits or channels could be tubes through which a first medium (i.e. either blood or air) flows whilst the space around the tubes provides a conduit for the flow of the second medium.

In one aspect, the walls defining the conduits are gas-permeable membranes allowing oxygen and carbon dioxide to diffuse in opposite directions. The blood flows in one direction through the device. Air may flow in alternate directions (as in normal breathing) or in directions controlled by fluidic logic. The total mass-exchange area is a fraction of the area found in the natural human lung of a living patient (e.g. about 5 to 20 square metres compared to about 100 square metres for a typical human lung). However, it is much larger than is employed in conventional blood oxygenators as used as part of heart/lung devices for thoracic surgery, which typically provide less than one square metre of surface area.

The solution now provided by the Applicant may in one aspect, be implemented as a prosthetic lung comprising an elastic bellows and at least one mass exchange apparatus herein with or without fluidic logic to provide a greater proportion of the flow in a desired direction.

In another aspect, the solution may be implemented as an external respiratory aid to augment lung function consisting of at least one mass exchange apparatus herein and auxiliary equipment to pump air and blood through the device. Applicant has appreciated that such an external respiratory aid is particularly suitable for use in the treatment of people with Acute Respiratory Infection. The WHO estimates that about 4 million people a year die from this cause. In a further aspect, the solution may be implemented as an intermediate device, in which part of the device is internal to the patient and part externally located.

It is noted that Applicant's solution makes use of an air supply and does not therefore require the use of an oxygen supply (i.e. pure or concentrated oxygen supply), which otherwise necessitates the use of weighty and bulky oxygen cylinders or oxygen generators. Applicant's solution may therefore be assembled in a lighter and more compact form than apparatus (e.g. conventional blood oxygenators) that rely on an oxygen supply.

It is an object of the present invention to provide a prosthetic lung for use in a human body. It is another object of the present invention to provide an external respiratory aid for use external to a human body. It is a further object of the present invention to provide an intermediate respiratory aid for use part internal to a human body and part external thereto.

### SUMMARY OF THE INVENTION

According to a first aspect of the present invention there is provided a mass exchange apparatus for use in blood/air mass exchange comprising
(a) plural blood flow conduits for defining blood flow;
(b) plural air flow conduits for defining air flow;
   wherein said plural air flow conduits and said plural blood flow conduits at least partially comprise gas-permeable membrane material, and the conduits are arranged relative to each other such as to enable transfer of oxygen from the air to the blood and transfer of carbon dioxide from the blood to the air through said membrane material.

Within the apparatus, the blood and air do not directly come into contact.

It will be appreciated that the walls defining the blood flow and air flow conduits may be separately formed and arranged relative to each other to enable the necessary exchange of air and carbon dioxide.

In one aspect, the blood and air flow conduits share at least some common walls, again with the arrangement selected to enable the necessary exchange of air and carbon dioxide.

Suitably, the blood flow conduits and/or air flow conduits have a diameter (or cross-section of non-circular conduit) of less than 0.5 mm.

The walls defining the blood and air flow conduits may comprise conventional materials (e.g. polymers) or composite materials. A composite material may comprise of two components, a first material component of the composite provides physical strength and a second material component provides gas and/or liquid permeability.

Suitable materials for the walls include those described in European Patent Application No. 1,297,855 in the name of Dainippon Ink & Chemicals. Thus, the materials suitably comprise a hollow fibre membrane comprising poly-4-methylpentene-1 and having an oxygen permeation rate Q(O₂) at 25° C. of from 1×10⁻⁶ to 3×10⁻³ (cm³(STP)/cm².sec.cmHg) and an ethanol flux of from 0.1 to 100 ml/min.m², wherein said membrane has (e.g. in the side of the blood flow) a surface comprising an ionic complex derived from:
quaternary aliphatic alkylammonium salts; and
heparin or a heparin derivative, and
   wherein said quaternary alkylammonium salts comprise a quaternary aliphatic alkylammonium salt having from 22 to 26 carbon atoms in total and a quaternary aliphatic alkylammonium salt having from 37 to 40 carbon atoms in total.

Suitably, the quaternary alkylammonium salt comprises from 5 to 35% by weight of a quaternary aliphatic alkylammonium salt having from 22 to 26 carbon atoms in total and from 65 to 95% by weight of a quaternary aliphatic alkylammonium salt having from 37 to 40 carbon atoms in total.

Suitably, the quaternary aliphatic alkylammonium salt comprises a dimethyididodecylammonium salt or a dimethyidioctadecylammonium salt.

Suitably, air and blood flows are arranged such as to provide blood oxygen/carbon dioxide relationships similar to those for natural respiration.

In one aspect, the air flow pattern is a combination of counter-current to the blood flow and co-current to the blood flow and may include recycled air flow.

In another aspect, the air flow is mainly counter-current (i.e. in the opposite flow sense) to the blood flow.

The blood/air mass exchange apparatus of the present invention is a counter-diffusion device that functions to transfer oxygen from the air into the blood and carbon dioxide from the blood to the air. In the air/blood mass exchange apparatus, blood and air flow in alternate channels between a series of plates that are separated by a small distance. Suitably, the spacing between the plates is less than 0.5 millimetres, preferably from 0.2 to 0.05 millimetres.

The plates are gas-permeable membranes allowing oxygen and carbon dioxide to diffuse in opposite directions. Alternative arrangements with channels or tubes of various cross-sections are possible. The blood flows in a first direction through the apparatus. Air may flow in alternate directions (as in normal breathing); counter-current to the airflow; intermittently counter-current; co-current or intermittently co-current to the airflow. The total mass-exchange area is a fraction of the area found in a living human lung. Thus, it is expected to be of the order of from 5 to 20 square metres, for example about 10 square metres compared to 100 square metres that is typically found in a human lung. Where more than one mass exchange apparatus herein, are used together the total mass exchange area is divided between the apparatus (e.g. where two apparatus are used in tandem, the total mass exchange area provided by these two in combination should be from 5 to 20 square metres).

A total mass-exchange area of from 5 to 20 square metres is a multiple of the area conventionally found in blood oxygenators used as part of heart/lung devices for thoracic surgery. Such blood oxygenators typically provide less than one square metre of surface area. The apparatus herein typically employs a larger area because it employs air (giving a lower mass transfer driving force) instead of oxygen, and is intended for medium to long-term use (days to years) by a conscious, mobile patient. Natural air is employed to give light weight and mobility rather than requiring the use of enhanced oxygen concentrations that require an oxygen supply (e.g. provided as a weighty oxygen cylinder). Blood oxygenators use oxygen as the gas phase. They are normally used over limited periods (of hours) with unconscious patients with low metabolic rates, often at lowered temperatures to reduce metabolic rates further.

Suitably, the apparatus herein includes a sensor (e.g. within a controller) for sensing a patient's demand for oxygen. In one aspect, the sensor detects the pulse rate of a patient, which tends to reflect patient demand for oxygen.

The sensor typically communicates with a controller that controls the exchange rate (e.g. increasing the exchange rate when more oxygen is needed, and decreasing the exchange rate when less oxygen is needed). The sensor is typically, an electronic sensor and communication with the controller may be via wired or wireless electronic transmission means.

In one aspect, the mass exchange apparatus of the present invention is incorporated into a prosthetic lung comprising bellows or air sac means (e.g. in the form of an elastic air sac) and at least one mass exchange apparatus herein. The bellows act such as to supply (e.g. draw or drive) air flow through the air flow conduits.

In another aspect, the mass exchange apparatus of the present invention is incorporated into an external respiratory aid to augment lung function comprising the mass exchange apparatus and auxiliary equipment to pump air and blood through the device.

Thus, according to another aspect of the present invention there is provided a respiratory aid apparatus for external connection to a patient comprising (a) at least one mass exchange apparatus as described herein; (b) an air pump for pumping air through said air conduits; and (c) a blood pump for pumping blood through said blood conduits.

Suitably, the respiratory aid apparatus comprises two mass exchange apparatus arranged in parallel fashion. This arrangement has benefits including the facility to replace one mass exchange apparatus whilst the other is still operational (e.g. still functioning).

The external respiratory aid apparatus suitably includes a sensor and/or controller, as described above. The controller is designed to ensure that the blood and/or air flow rates are adjusted to respond to the blood flow rate in the patient. The controller is required for a conscious, mobile patient whose heart (and breathing) rate responds to their level of activity.

The external respiratory aid apparatus suitably incorporates tubing to extract oxygen-depleted, high carbon-dioxide, blood from the patient and return oxygenated blood, with low carbon dioxide. Separate tubes may extract the blood and return it. Alternatively, the extraction and return tubes may be joined concentrically to simplify fitting the device and to extract and return blood from adjacent positions (for example, in the vena cava system). In this way, no vein or artery would suffer depleted blood flow. Particularly, the heart would experience a full flow of oxygenated blood.

Suitably, the external respiratory aid apparatus herein, allows the option of recycling some of the air through the mass exchange apparatus to increase the carbon dioxide concentration and hence provide a means of separately controlling oxygen and carbon dioxide concentrations in the blood.

In one use aspect, the external respiratory aid is arranged to allow the option of blood extraction and return through a single entry point in a vein of a patient. Thus, input tubing to the blood pump is arranged to provide blood extraction and return via the desired single entry point. This mode of use simplifies the clinical procedure.

Suitably, the external respiratory aid apparatus is provided with short connecting lines (e.g. tubes of length less than 1 metre, preferably less than 0.5 metres) for connecting to the patient to provide the desired air and blood flows. Short connecting lines are preferred because heat loss is thereby minimized, thus reducing any risk of hypothermia. Alternatively heated lines may be employed (e.g. using heat exchange with the body), but this approach adds complexity.

Suitably, the respiratory aid apparatus is arranged such that extracted blood undergoes counter-current heat transfer with returned blood. This arrangement desirably minimizes any temperature fall in the blood extracted from the body and returned after mass exchange.

Suitably, the respiratory aid apparatus additionally comprises an air filter for filtering the air. A HEPA filter is an example of a suitable air filter.

Optionally, where it is desired to minimize the loss of water vapour from the patient, the respiratory aid apparatus additionally comprises a humidifier for humidifying the air. Optimally, humidified air is directed to the mass exchange apparatus at near blood temperature.

Suitably, the respiratory aid apparatus additionally comprises a heat exchanger. Suitably, the air flow is arranged to pass through a heat exchanger that uses body-heat to pre-heat the air to near body-temperature. The heat exchanger may consist of one or more flexible tubes or conduits that are arranged into a sheet that is placed against the body of a patient and insulated on the side away from the body of a patient.

In a further aspect, the mass exchange apparatus of the present invention is incorporated into an intermediate respiratory aid for placing inside the body of a patient (without removing the lungs), such that the blood is pumped through the mass exchange apparatus by the natural circulatory system (ultimately the heart) of the patient. The air supply is suitably, external. The mass exchange apparatus is suitably arranged to connect directly to a vein, for example of the vena cava system, of a patient. The intermediate respiratory aid eliminates the necessity for the blood pump of the external respiratory aid. The device could take all, or part of the blood flow. The air would be pumped from outside the body, as for the external respiratory aid. As for the external respiratory aid, the flow pattern and relative flow rates would suitably be adjusted such that the natural carbon dioxide/oxygen relationship was mimicked. Desirably, located outside the body of a patient, there is a HEPA filter between the pump and the entry point of the tube into the body. The air exhaust from the exchanger is conducted outside the body, where it is discharged to atmosphere.

Thus, according to another aspect of the present invention there is provided an intermediate respiratory aid apparatus for internal connection to a patient comprising (a) at least one mass exchange apparatus as described herein; and (b) an air pump for pumping air through said air conduits.

Optionally, the intermediate respiratory aid has a sensor and controller to control the air pumping rate (and possible recycle rate) to give desired oxygen and carbon dioxide concentrations in response to increased metabolic oxygen demand.

The prosthetic lung, external respiratory aid and intermediate respiratory aid, each have a distinct purpose compared to a heart/lung machine in that they are intended to be permanently connected to a patient who is conscious and mobile. To achieve this goal, they are designed to be robust, lightweight and portable.

The small size of the mass exchange apparatus is possible because:

1. Fresh air is contacted directly with the membranes. This arrangement increases the driving force (and hence rate) of mass transfer by a factor approaching five compared to the human lung in which the air sacs are at the end of long narrow passageways within the lung.

2. The velocity of the air through the mass exchange apparatus is much higher than the velocity at the mass-transfer surface in the human lung. In a human lung, the relative velocity is almost zero in the air sacs where transfer takes place. An increased relative velocity increases the mass transfer coefficient so that the total mass transfer rate per unit area may be an order of magnitude greater than in the human lung.

The mass-exchange apparatus of the present invention is suitably designed for long-term, maintenance-free operation. The straight passages, with relatively high air velocity are suitably designed to be self-clearing. This self-cleaning characteristic is important because prosthetic lungs will not have the ciliary action found in living lungs.

The mass-exchange apparatus of the present invention suitably employs indirect gas/liquid contact.

Applicant has appreciated that counter-current air flow maximizes mass transfer rates in an exchanger of a given area. However, counter-current flow disproportionately increases the efficiency of carbon dioxide mass transfer. Accordingly, co-current flow and recycle may be included to match the natural carbon dioxide/oxygen relationship in the blood. In this way, the body's natural respiratory control mechanisms operate normally. Normal operation of the control mechanisms (primarily sensing carbon dioxide levels) has two benefits. The first benefit is that the natural control mechanisms for the metabolic system as a whole operate normally and correctly. The second benefit is that any external controller can take advantage of natural responses (such as increased heart rate) to maintain correct blood oxygen and carbon dioxide levels without necessarily employing recourse to direct measurement of blood gas compositions.

Suitably, when the external respiratory aid apparatus takes only a fraction of the blood flow, mass transfer is maximized by employing counter-current air flow. When larger blood flows are taken, for example with the intermediate respiratory aid, air flow patterns including co-current and recycle flow may be employed to mimic natural oxygen/carbon dioxide relationships in the blood.

For the prosthetic lung, fluidic logic is a possible method of achieving the desired flow patterns throughout the breathing cycle. In this aspect, fluidics replaces the electronic logic anticipated for the external and intermediate devices. A number of known fluidic devices have no moving parts so that very low maintenance would be required even for this more complex flow arrangement.

Prosthetic Lung

In the prosthetic lung aspect of the present invention, the mass exchange apparatus is connected directly to the blood circulation, so that the heart pumps blood through it in the same way that it does natural lungs. The natural lungs are removed and each lung replaced with an elastic air sac (or bellows). The bellows are placed in the pleural cavity from which the lungs have been removed. The natural breathing action expands and contracts the bellows so that they draw air through the mass exchange apparatus. No blood circulates through the bellows, which can be designed to be rugged and maintenance-free.

To provide additional protection for the mass exchange apparatus, it may be installed within the bellows. The bellows typically occupy 5 litres each and deliver between 0.5 and 2 litres of air on each breath. Thus, there remains sufficient space within the bellows to install a mass exchange apparatus for each “lung”. In order to accommodate a mass exchange apparatus in each lung-space, the total volume of each mass exchange apparatus must be less than about 3 litres. From a weight viewpoint, the aim will be to provide sufficient mass transfer surface in a significantly smaller volume. The bellows either will connect directly to the trachea (when there will be an engineered division between the two lungs) or will connect to the bronchi after they have divided from the trachea.

Benefits provided by a prosthetic lung of this form include:

1. There are no moving parts (other than elastic expansion and contraction of a balloon-like bellows). The heart provides the blood circulation. The patient's own breathing action provides the required airflow.

2. There is no requirement for control equipment. The patient's natural reflexes will cause the heart and breathing rate to match their oxygen requirements. The natural control action senses carbon-dioxide levels in blood. If it is high, respiration increases; if it is low, respiration decreases. It follows that ultra-precise design is not required. The body will automatically adjust how hard it works to the efficiency of the prosthetic lungs. (The same behaviour occurs in nature if living lungs are damaged). If efficiency deteriorates over the years, the body just works harder to accommodate the changes.

3 Pre-warmed humidified air is provided by the body's natural systems.

There are several ways of fitting the mass exchange apparatus into the lung-bellows. It may be simply sealed so that all the air comes through the device when the patient breathes in and all the air goes out through the device when the patient breathes out. The lungs may be designed for counter-current flow on the “in” breath to maximize mass transfer rates. Alternatively, the lung may be designed for co-current flow on the “in” breath, in order to reduce the efficiency of carbon dioxide transfer relative to oxygen. As a further alternative, fluidic logic may be employed to generate suitable air flow patterns to mimic the natural relationship between oxygen and carbon dioxide in respiration through healthy lungs. The low pressure-drop fluidic device could be mechanical or have no moving parts.

The form of the prosthetic lung in accord with the present invention has similarities with the lungs of birds. Birds breathe by, in effect, operating a bellows that draws air through a rigid matrix in which the counter-diffusion takes place. In the context of the prosthetic lung, this arrangement has the advantage that the matrix can be constructed from a simple arrangement of straight conduits (e.g. in plate form). For example, the matrix could be constructed from several hundred (up to a few thousand) thin parallel sheets. Blood and air would flow through alternate sheets, similar to a plate and frame heat exchanger. A similar effect could be achieved with an arrangement of fine tubes (either circular, or non-circular in cross-section). Either the blood or the air could flow through the tubes, depending on the detailed design. This construction (either sheets or tubes) solves several problems. First, sizes are within achievable robust engineering construction limits (materials can be around 0.1 mm thickness). Secondly, straight flow channels can allow self-clearing without ciliary action. Thirdly, the relatively high air velocity and oxygen concentration through the channels gives enhanced mass exchange requiring a smaller surface area for the same lung performance. These prosthetic lungs would have no moving parts, and no control mechanism would be required. The body's natural control action would apply. Thus, the brain senses blood carbon dioxide concentration and causes the heart and breathing rate to respond appropriately. There is the further benefit that the conduits could be mass-produced and assembled to meet the size requirements of individual patients.

The major performance differences between the proposed prosthetic lung and known heart-lung machines are that the prosthetic lung has small size for ready portability; a maintenance-free design life of years rather than hours; and no intrinsic requirement for “heart” action.

External Respiratory Aid Apparatus

In the external respiratory aid apparatus aspect of the present invention, part of the oxygen-depleted blood in the veins approaching the heart of the patient is diverted and taken out of the body through a tube inserted in the blood vessel. The diverted blood is passed through an externally located mass exchange apparatus. The blood is returned to the main arteries leaving the heart. Alternative extraction and return points are possible. For example, the blood could be taken from the veins before the heart and returned to the veins at a later point, still before the heart. In this way, the heart does not have to work with depleted blood flow or deficient oxygen supply. A further benefit of this arrangement is that the extraction and return tubes could be joined to require only one entry point into the vein system. For example, the tubes could be concentric, with the return tube inside the extraction tube. The alternative of placing the extraction and return points between the heart and lungs would make the closest match to the performance of natural fully functioning lungs. However, it is suspected that the clinical operation to insert tubes at that point would be prohibitively complex.

The heart itself would probably be incapable of driving a flow-divider that sent a proportion of the blood through the external respiratory aid. A peristaltic pump or other device designed not to damage the blood therefore typically pumps the extracted blood through the mass exchange apparatus. A small fan is suitably used to drive air through the exchanger. Such an external respiratory aid is clearly heavier than a prosthetic lung because it requires a pump, a fan and a power source. The total device (mass exchange apparatus plus pump and power source) would weigh at least a fraction of a kilogram, and might weigh several kilograms. However, even at several kilograms it would still be sufficiently portable to enable to the patient to exercise and achieve a level of fitness that would not otherwise be possible.

Taking the blood flow outside the body to the external respiratory aid apparatus gives greater risk of infection. The apparatus is also bulkier and more complex. However, there will be a range of applications in which an external respiratory aid is preferred. For example, the lung condition may be reversible (such as occurs with Acute Respiratory Infection). It would be counter-productive to remove a potentially healthy lung. In some circumstances the device might replace a heart-lung machine.

In normal applications, it is anticipated that only part of the blood supply will go through the mass exchange apparatus. This division is made because it leaves no blood vessels entirely devoid of flowing blood; and it leaves the normal mammalian control functions operational. Thus, if carbon-dioxide levels rise, the patient's heart and lungs will work harder. Unless lung function is completely lost, such action will reduce carbon dioxide and increase oxygen. In this way, the patient will avoid the confusion of a non-functioning respiratory control system. At the cost of additional complexity, blood flow could be monitored and the blood and air flow though the external respiratory aid automatically adjusted according to rate. In this way, an approximately constant fraction of the blood flow would be diverted through the external respiratory aid, and desired blood oxygen and carbon dioxide concentrations achieved. This control action is important where the patient's own lungs are severely compromised. Without control, there is risk of extracting a flow greater than that in the relevant vein, resulting in damage through reverse flow in the vein. Furthermore, without control, the patient may sense a reversal of the normal physiological responses. Thus, as the heart beats faster, and the blood flow increases, the fixed flow of oxygenated blood from the external exchanger would be diluted by a larger flow. The resulting mixed blood flow would have lower oxygen and higher carbon dioxide concentration. This response could confuse the patient's natural control system that expects oxygen levels to rise and carbon dioxide levels to fall when the heart beats faster and the patient breathes harder. Control (e.g. by means of a suitable sensor/controller) would restore the normal response to heart rate and breathing. The invention herein, includes the option of co-current air flow and/or recycle of part of the air through the external exchanger. Use of co-current air flow and/or air recycle increases carbon dioxide concentration proportionately more than the decrease in oxygen concentration. Adjusting total air flow and recycle rates separately, enables the blood concentrations of carbon dioxide and oxygen to be independently adjusted. The required relationships are easily programmed into an automatic controller that only needs to sense one measure of metabolic oxygen demand.

The provision of an external respiratory aid that removes carbon dioxide from the blood may permit additional treatments. For example, a number of lung infections result from bacteria that are averse to high oxygen concentrations. In such a situation, there is no benefit in breathing higher levels of oxygen (for example, beyond 40%) because the defective lungs cannot get rid of the excess carbon dioxide. The provision of an external, auxiliary breathing-device would overcome this constraint. It is this kind of thinking that allows the possibility that the oxygenated blood might be returned upstream of the lungs.

Intermediate Respiratory Aid Apparatus.

For longer-term use, the external respiratory aid can be replaced by an intermediate system in which the mass exchanger is within the body. The intermediate system eliminates the necessity for a blood pump and is less vulnerable to damage.

Use Aspects

The mass exchange apparatus, prosthetic lung and respiratory aid devices herein are suitable for use with a human or animal (particularly mammalian) subject. Installation and/or use is typically under the control of a physician or veterinary surgeon.

No previous apparatus or device has been described that allows lung function to be augmented or replaced for extended periods with the patient mobile and conscious, and that makes use of natural air, unenriched with oxygen.

### BRIEF DESCRIPTION OF THE DRAWINGS

The present invention will now be described further with reference to the accompanying drawings, in which:

FIG. 1 shows a schematic representation of an air/blood mass exchange apparatus herein;

FIG. 2 shows a schematic sectional representation of a prosthetic lung herein within the body of a patient;

FIG. 3 shows a schematic representation of an external respiratory aid herein as suitable for connection to a patient;

FIG. 4 shows a schematic representation of a possible extraction/return system for the external respiratory aid herein; and

FIG. 5 illustrates a schematic representation of an intermediate respiratory aid apparatus herein.

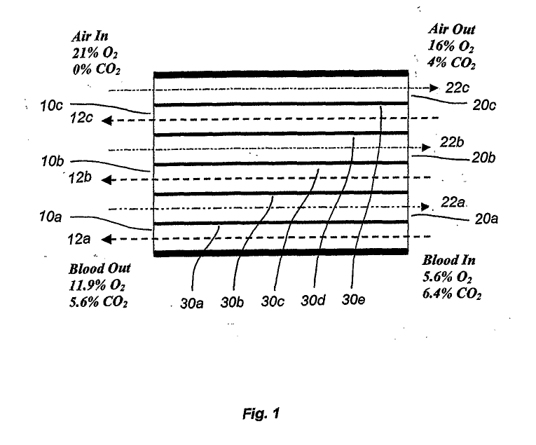

### Referring now to the drawings, FIG. 1 illustrates an air/blood mass exchange apparatus herein comprising plural blood flow conduits 10a to 10c for defining blood flow 12a to 12c; and plural air flow conduits 20a to 20c for defining air flow 22a to 22c. It may be seen that the blood 12a-c and air flow 22a-c is in alternate channels defined by a series of plates 30a-e separated by less than 0.5 millimetres. Whilst for the purposes of representation, FIG. 1 shows a relatively small number of channels it will be appreciated that the actual apparatus will comprise several thousand channels to give an overall mass transfer area of from 5 to 20 square metres.

The blood flows in a first direction 12a-c through the apparatus. As shown, the air flows in a second direction 22a-c counter to the first direction. In aspects, air may flow in alternate directions (as in normal breathing), counter-current to the air flow, or intermittently counter-current to the air flow. Particularly, the air flow 22a-c may be arranged to be a combination of air flow 22a-c that is counter-current to the blood flow 12a-c and air flow 22a-c that is co-current to the blood flow 12a-c. The plates 30a-e are gas-permeable membranes that enable transfer of oxygen from the air to the blood and transfer of carbon dioxide from the blood to the air through said membrane material. FIG. 1 also recites typical molar (or volumetric) concentrations for oxygen and carbon dioxide. In aspects, the apparatus may additionally be provided with flow headers and dividers in accord with conventional heat exchanger design practice.

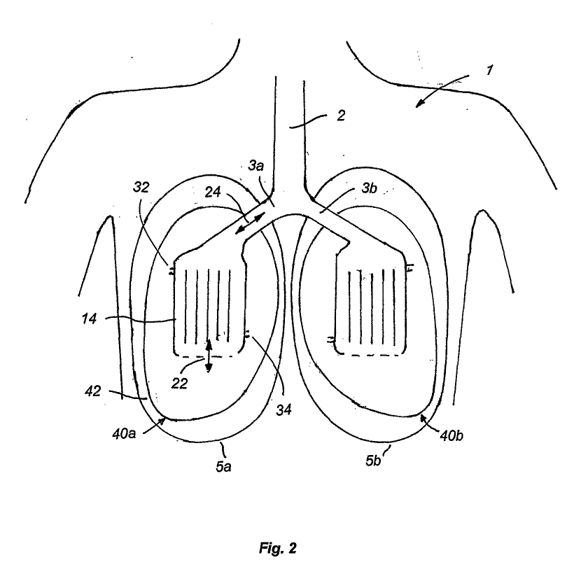

FIG. 2 illustrates in cutaway view a patient 1 having a trachea 2 leading to the left and right bronchi 3a, 3b. Both of the patient's lungs have been removed and within the left and right pleural cavity 5a, 5b there has been ‘transplanted’ a prosthetic lung 40a, 40b in accord with the present invention. The structure of the left-hand prosthetic lung 40a is now described in detail (that of the right hand prosthesis is a mirror image).

The prosthetic lung 40a comprises an elastic air sac 42 sized and shaped for receipt by the lung cavity 5a. Within the elastic air sac 42 there is provided an air/blood mass exchange apparatus 14 herein comprising plural blood flow conduits for defining blood flow and plural air flow conduits for defining air flow (detail not shown, but corresponds to that of FIG. 1). To create the air flow, air inlet 22 leads from the bellows defined by the air sac 42 and air outlet 24 leads into the patient's left bronchus 3a. In use, the patient will control air flow by means of the same instinctive chest motion that drives living lungs. Thus, the bellows 42 will be alternately expanded and compressed. The bellows 42 will contract under their own elasticity (as do living lungs) and they will be expanded by muscular action. During the contraction part of the breathing cycle, the bellows 42 pumps air through the inlet 22 to the mass exchange apparatus 14 and thence to the outlet 24. During the lung expansion part of the cycle, the pressure within the bellows 42 will fall below atmospheric pressure causing air to rush in through the outlet 24 and the exchanger 14 to the inlet 22 and bellows. Thus, two way air flow is enabled.

In the absence of fluidic logic, the following flow patterns are possible. The inlet breath may be counter-current to the blood flow 12a-c, and the outlet breath co-current. This arrangement maximizes mass transfer rates. Alternatively, the inlet breath may be co-current with the blood flow 12a-c, and the outer breath counter-current. This arrangement disproportionately reduces the efficiency of carbon dioxide mass transfer. Mass transfer will take place in the mass transfer apparatus 14 during both parts of the cycle, but will be more effective on the “in” breath. As a further alternative, the air flow may be controlled by fluidic switches so that air-flow patterns are achieved that give O₂/CO₂ relationships more closely mimicking the natural relationships. In this case, it might be required to divide the mass exchange apparatus into parts with distinct flow patterns in each part.

The patient's blood flows into the mass exchange apparatus 14 by means of blood inlet 32 and exits via blood outlet 34. It will be appreciated that the blood flow inlet 32 and outlet 34 will be connected to the patient's blood supply and that flow will be governed by the pumping action of the patient's heart (not shown). The flow headers to divide the fluid flows between the channels and to keep the two fluids separate will be similar to those in a conventional heat exchanger, and are not illustrated.

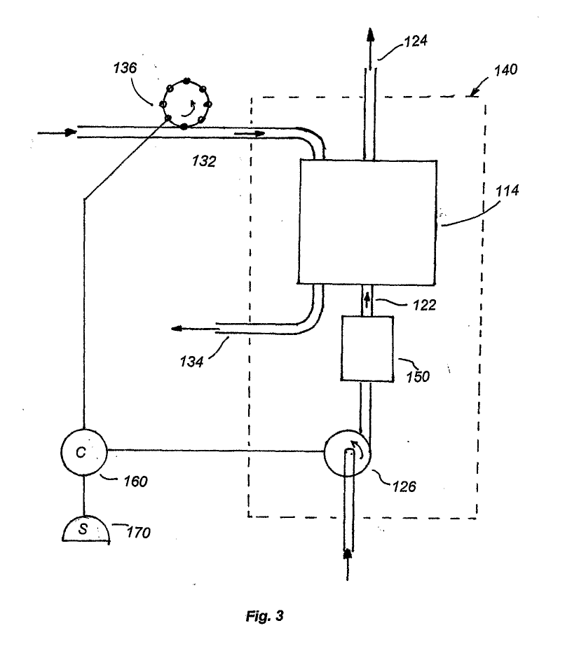

FIG. 3 illustrates an external respiratory aid apparatus herein shown in cutaway view. The external respiratory aid 140 comprises an air/blood mass exchange apparatus 114 herein connected up to air and blood flow apparatus. Whilst in the embodiment shown in FIG. 3, there is a single mass transfer apparatus, variations are envisaged in which two mass exchange apparatus arranged in parallel fashion.

To create the air flow, air inlet 122 leads from pump 126 (e.g. in the form of a fan) to direct air in a first direction through the mass exchange apparatus 114 (e.g. having the detailed form of that mass exchange apparatus of FIG. 1) from which it exits at air outlet 124. In use, the air flow is controlled by suitable control of the air pump 126. The patient's blood flows into the mass exchange apparatus 114 by means of blood inlet 132 and exits via blood outlet 134. It will be appreciated that the blood flow inlet 132 and outlet 134 are connected to the patient's blood supply.

Blood flow is governed by the pumping action of blood flow pump 136. The pump is designed to minimize damage to the circulating blood flow. A number of pump designs are possible, and a peristaltic pump is illustrated. The respiratory aid apparatus 140 is also connected up to an air filter 150 that may also act as a humidifier. Optionally, the air can also be pre-heated with a simple heat exchanger in contact with the body. As illustrated, the blood flows in a first direction through the apparatus 140 and the air flows in a second direction counter to the direction of blood flow. As described below in “Mass transfer in respiratory aids and prosthetic lungs”, control of carbon dioxide levels may be important, when the alternative of co-current flow may be advantageous, as may the provision of partial air recycle.

The air flow pump 126 and blood flow pump 136 may be seen to communicate with controller 160, which in turn communicates with sensor 170. The sensor 170 is arranged to sense the oxygen demand of a patient (not shown). Oxygen demand may be sensed indirectly through, for example, measuring pulse rate. The controller 160 controls the pumping action of both pumps 126, 136 in response to signals received from the sensor 160, and hence acts to control the rate of blood/air mass exchange.

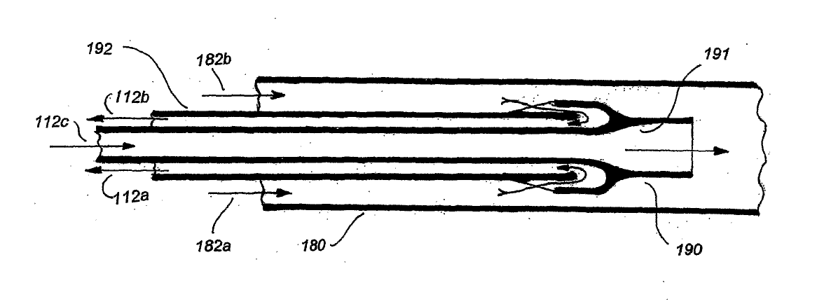

Desirably, the input tubing 132 to the blood pumpl36 is arranged to provide blood extraction and return via a single entry point in a vein of a patient. An extraction head, which is suitable for installation by use of concentric input tubing 132 is illustrated in FIG. 4 herein.

Referring now to FIG. 4, the vein 180 of a patient receives first 190 and second 192 concentric tubes. The tubing is arranged such that the blood flow 182a-b within the vein is counter to the blood flow 112a, 112b to the mass exchange apparatus, which flows in the outer concentric tube 192. In turn, the blood flow from the mass exchange apparatus 112c within the inner concentric tube 190 flows counter to the blood flow 112a, 112b to the mass exchange apparatus.

In the design shown in FIG. 4, the extraction point is immediately upstream of the return point. The external surface of the extraction/return head is designed so that the device can be inserted into the vein 180 at a convenient point and then threaded to a suitable point, for example in the vena cava system. The design also allows withdrawal of the device without major surgery. In this way, the use of the external respiratory aid is easily reversible. A similar design applies for the case where the extraction and return channels are side-by-side, rather then concentric.

At the point of extraction, the outer tube (annulus) may have holes or a mesh through which the blood is extracted. The extracted blood 112a, 112b reverses direction to flow through the extraction tube. The returned blood 112c is arranged to flow in the same direction as the blood 182a, 182b in the vein from which it is extracted. By suitably tapering 191 the inner concentric tube 190 at the return point, the returned flow can mingle with the residual flow in the vein with both flows at approximately the same average velocity.

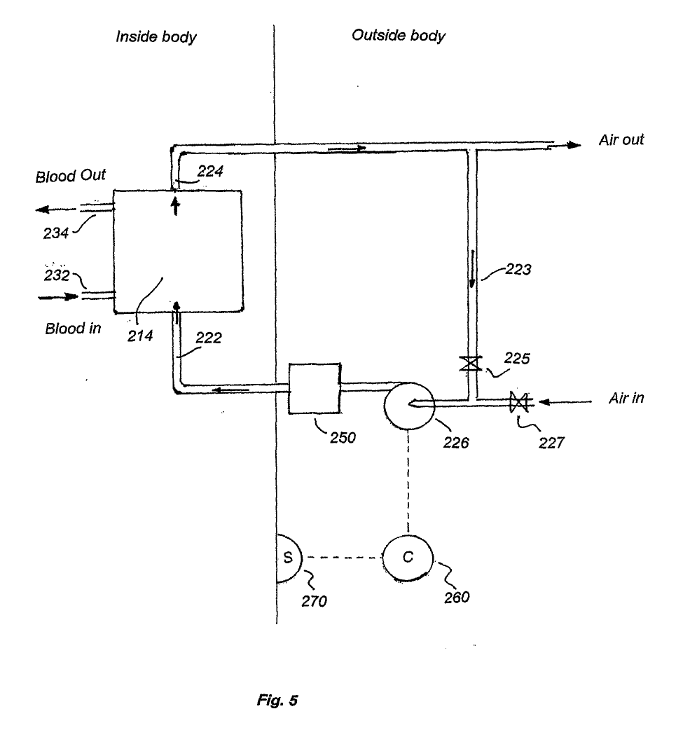

FIG. 5 illustrates an intermediate respiratory aid apparatus herein shown in cutaway view. Part of the apparatus locates within the body of a patient and part locates outside of the body.

The intermediate respiratory aid comprises an air/blood mass exchange apparatus 214 herein (e.g. having the detailed form that apparatus of FIG. 1) connected up to air flow apparatus. The mass exchange apparatus 214 is arranged to connect directly with a vein of a patient such that the blood flow is provided by the action of the patient's own heart. To create the air flow, air inlet 222 leads from pump 226 (e.g. in the form of a fan) to direct air in a first direction through the mass exchange apparatus 214 from which it exits at air outlet 224. In use, the air flow is controlled by suitable control of the air pump 226. Blood flows through the mass exchange apparatus 214 (in response to the action of the patient's heart) by means of blood inlet 232 and exits via blood outlet 234.

The air flow is delivered through a HEPA filter to clean the air before delivering it to the mass exchange apparatus 214. It will also be seen that recycling channel 223 is used to recycle air from the outlet 223. Restrictors 225 and 227 are employed to control the amount of recycled air employed that is pumped back to the air inlet 222. As for the external device, the air feed may also be humidified and pre-heated if required.

The air flow pump 226 may be seen to communicate with controller 260, which in turn communicates with sensor 270. The sensor 270 is arranged to sense the pulse rate of a patient (not shown), which rate is indicative of the patient's demand for oxygen. The controller 260 controls the pumping action of the air pump 226 and hence controls the overall rate of blood/air mass exchange.

The Function of the Human Lung.

In engineering terms, the performance of the human lung can be characterized in terms of its two input streams and its two output streams. The two input streams are atmospheric air (cleaned, humidified and adjusted to body temperature by passage through the nose etc) and (venous) blood depleted in oxygen. The two output streams are exhaled air and oxygenated (arterial) blood. We are also interested in the tracheal air composition in the air sacs; this air contacts the blood via the mass transfer membranes and provides the driving force for the counter-diffusion. The lung performance is determined by the transport equation:
m=UAΔc (1)

In equation (1), m is the mass transfer rate (moles/second or grams/second), U is the overall mass transfer coefficient, A is the interfacial area for mass transfer and Δc is the concentration difference driving the mass transfer. Equation (1) applies both to oxygen and to carbon dioxide by inserting the appropriate driving forces and mass transfer coefficients.

In order to compute Δc, we need to know the concentrations in air of oxygen and carbon dioxide in equilibrium with the various blood streams rather than the actual concentrations in the blood streams. Note that there is a highly non-linear relationship between blood oxygen concentration and equilibrium gas-phase concentration. These equilibrium gas-phase concentrations are given in the Table 1. Concentrations are molar or volumetric. (The percentage concentration figures also closely approximate the numerical values of the partial pressures measured in kPa).

TABLE 1
Oxygen and Carbon Dioxide Concentrations.

| | Oxygen | CO₂ |
|---|---|---|
| Stream | Concentration (%) | Concentration (%) |
| Atmospheric Air Input | 21 | 0.0 |
| Venous Blood Input | 5.6 | 6.4 |
| Exhaled Air Output | 16 | 4 |
| Arterial Blood Output | 11.9 | 5.6 |
| Alveolar Air | 14 | 5.6 |

Some of the values vary considerably from individual to individual. However, it is seen that, even with alveolar air, there is a minimum driving force of about 2% (14-11.9) to drive the mass transport of oxygen from the air into the (oxygenated) arterial blood. There is a very small driving force to drive the mass transport of carbon dioxide from the arterial blood to the alveolar air. Clearly, there is a much larger initial driving force as the air contacts the returning venous blood, but the driving force declines as the blood oxygen concentration rises and carbon dioxide level falls.

The Structure of the Human Lung

The trachea divides into two bronchi to feed the two lungs. These bronchi divide and divide again until, at the alveoli, they terminate in about 750 million small air sacs. At this point, gases exchange between air and blood through the thin membranes forming the sacs. The maximum volume of air that can be accommodated in the lungs is typically 5 litres (varying from person to person in a range from about 3 to 7 litres). The total space in the lung cavity is typically less than 10 litres. In normal breathing, about half a litre of air is respired per breath. The maximum that can be respired per breath is about 4 times the normal amount. The lungs serve the purpose of transferring oxygen from the air to the blood in order to replenish that consumed by metabolic processes. Equally, they serve the purpose of transferring carbon dioxide from the blood to the air to discharge that produced by the metabolic processes. The surface area for this exchange is about 100 m². The lungs are elastic so that they contract when not drawn out by the act of breathing. The inner surface of the lungs is furnished with cilia that enable debris to be transported out and the surfaces kept clean. The lungs share the important characteristic of all living organs that, within limits, they can repair themselves. Thus, even if the repair involves scarring, minor injuries will be repaired. For example, blood does not leak into the inside of the lungs or into the space surrounding the lungs (the pleural cavity). Similarly, air does air leak through the lungs into the cavity. Additionally, the pleural cavity is lubricated to avoid damage to the lungs during the normal act of breathing. The human body includes an automatic control system that adjusts the rate and depth of breathing to a level adequate to supply oxygen and remove carbon dioxide. The system works primarily by detecting carbon dioxide levels in the blood. (A by-product of this control system is that we can easily detect when we are somewhere with a high carbon dioxide concentration, but do not easily detect when oxygen levels are depleted).

In lung disease, the effective size of the lungs is reduced. Reduced lung capacity as low as 30% only marginally affects normal life. Obviously, any form of strenuous exertion becomes impossible, but a person could live a more-or-less normal life with only minor symptoms. At 20% capacity, the person may not be wheelchair bound, but will only be able to walk a few yards at a time. They may require periods on increased oxygen, and during minor infections (for example, a cold) may need admission to hospital. They will be using bronchodilator drugs to squeeze extra capacity from their lungs and may be on other medication. Further reduction in lung capacity results in more severe symptoms that cannot be cured even by permanently breathing high oxygen concentrations. Although high oxygen concentrations enable more oxygen to get into the blood stream, the carbon dioxide produced by metabolic processing cannot be cleared. Oxygen is carried in the blood primarily as oxyhaemoglobin. Most carbon dioxide is carried in the blood as bicarbonate ions. However, about 20% is carried as carboxyhaemoglobin. Thus, carbon dioxide and oxygen compete for haemoglobin. It follows that high concentrations of carbon dioxide reduce the capacity of the blood to transport oxygen. The driving force for the metabolic processes (digestion of food, muscle activity etc) is then impaired because these all consume oxygen and produce carbon dioxide. Thus, the ability to maintain life-supporting metabolic processes is severely diminished. By 10% lung capacity, death is almost certain. A patient is likely to be placed on a lung transplant list if they are otherwise healthy, but are likely to have significantly less than 20% lung function within two years. (There is no clear-cut formula; clinical judgement is employed).

Mass Transfer in Respiratory Aids and Prosthetic Lungs.

In the natural lung, the overall mass transfer resistance is made up from four resistances to mass transfer. (Mass transfer resistance is the inverse of mass transfer coefficient). These are:
1) The gas-side resistance resulting from diffusion through the long, narrow bronchi and bronchioles. The difference between the alveolar composition and the input and output compositions gives a measure of this resistance.
2) The resistance to the gases diffusing through the membranes separating the air and blood in the air sacs.
3) The resistance to the gases diffusing through the liquid phase in the blood (and, in the case of oxygen, diffusing through the blood corpuscles to reach the haemoglobin).
4) The speed of chemical reaction in converting the oxygen to oxyhaemoglobin, and converting carbon dioxide to bicarbonate ions and carboxyhaemoglobin.

The combined resistances of steps (2) to (4) is seen to be very low for carbon dioxide because of the negligible driving force needed to transfer carbon dioxide from the alveolar air into the blood. Thus, for carbon dioxide, the total driving force for these three steps is (alveolar partial pressure)−(equilibrium partial pressure in the blood), namely 5.6−5.6≈0. For both gases, the gas-side resistance is indicated by the difference between the alveolar pressures and a mean of the inhaled and exhaled concentrations. For oxygen, the relevant differences are, for inhaled 21−14≈7 kPa, for exhaled 16−14≈2 kPa. For carbon dioxide, the relevant differences are, for inhaled 5.6−0≈5.6 kPa, for exhaled 5.6−4≈1.6 kPa. There is a simple calculation if we assume that the gas diffusivities are the same for carbon dioxide and for oxygen (including allowance for the drift effect). Thus, we would expect the carbon dioxide driving forces to be about 80% of the oxygen driving forces because the mass transfer rate of carbon dioxide is approximately 80% of that of oxygen. According to the figures in the table, the ratio is very close to this estimate for both the inhaled and exhaled air. In practice, the gas diffusivity of oxygen is about 25% higher than that of carbon dioxide, so that there must be compensating effects that make our approximate calculation so accurate.

The mass exchange apparatus that we propose have very small diffusion paths for the gas side (about half the diameter of the tubes). Gas diffusivities are between 10⁴ and 10⁵ times higher than liquid diffusivities. Thus, the mass exchange apparatus will almost eliminate the gas side resistance to mass transfer. The remaining resistance will be the resistances (2) to (4) in the list above. Thus, the mass transfer resistance for carbon dioxide is almost eliminated, whilst that for oxygen is decreased by a factor of between 2 and 7. It follows that the relative mass-transfer resistances differ considerably between the natural lungs and the mass exchange apparatus. The very low driving forces for carbon dioxide transfer may result in carbon dioxide partial pressures that are almost the same in the gas and liquid phases. In contrast, there is still significant resistance to mass transfer for oxygen, and the area will just be sufficient to give required mass transfer rates for oxygen concentrations in the range 16% to 21%. Thus, a simple counter-current mass exchange apparatus would give very low outlet blood carbon-dioxide levels (possibly less than 1%). These low values have a deleterious effect on the natural respiratory control mechanisms, which are expecting concentrations of the order 5%, and significantly higher than that for patients with established lung deficiency. It is for this reason that, even with the very small areas of heart/lung machine oxygenators, provision is made to add carbon dioxide during thoracic surgery. The natural relationship between oxygen and carbon dioxide concentrations can be restored by selecting a suitable flow pattern. For example, referring to the figures of Table 1, co-current flow would give an outlet blood carbon dioxide concentration in equilibrium with the outlet gas pressure of 4%. This value could be increased to the natural level of 5.6% by reducing the relative air flow rate by about 30%. Again referring to Table 1, the log mean driving force for oxygen transfer for counter-current flow is (10.4−9.1)/ln(10.4/9.1)=9.7. The corresponding figure for co-current flow is (15.4−4.1)/ln(15.4/4.1)=8.4. (The log mean is an approximate measure of the driving force averaged over the length of the mass exchange apparatus). Thus, switching to co-current flow decreases the mean driving force by about 14%. This reduction can be made good by a corresponding increase in mass transfer area. Thus, by appropriate choice of flow pattern (co-current, counter-current and/or air recycle) together with appropriate choice of relative air/blood flow rates, a natural relationship between blood oxygen and blood carbon dioxide levels can be restored. Precise matching is not required because the natural control mechanisms are self-adjusting over a range of lung performance levels. For an external (or partially external) device, a controller that sets the blood rate can also set the relative air/blood flow rates. For a prosthetic lung, the appropriate flow patterns may need to be set by fluidic logic.

Considerations for Designing a Prosthetic Lung

In designing a prosthetic lung, it is desirable that the solution does not restrict the normal movement of the patient. The apparatus desirably requires no maintenance for tens of years and fits into the lung cavity. The apparatus should also desirably have no motor or engineered control system, and be powered only by the normal movements of the chest and diaphragm.

It is appreciated to be difficult to design a readily manufacturable and robust prosthetic lung with anything approaching the surface area of the natural human lung. However, the human lung clearly utilises its vast surface area inefficiently. The air sacs are never flushed with atmospheric air. Fresh air thus mixes with stale air, which results in poor driving forces for mass transfer. Furthermore, the air in the sacs is stagnant which results in poor mass-transfer coefficients. Thus, referring to Equation (1), we see that, although A is large, the other terms are much smaller than they could be. As discussed above, the human lung is normally more than adequate for its purpose. Indeed, it can suffer major damage with only minor restrictions in function. It follows that evolution has no incentive to evolve a more efficient respiratory system for humans (or other mammals). We must look elsewhere for inspiration in designing prosthetic lungs. We need to consider creatures that have to sustain higher metabolic rates and thus need higher mass transfer rates. Such creatures would have lungs over-designed for human use, so that we would require only a fraction of their capacity. Birds need to sustain high metabolic rates to support flight. Evolution has driven their respiratory system to be more efficient than that of mammals. We will briefly describe the principles of a bird's “lung”. We will describe how its basic design could be adapted in a prosthetic lung.

Birds do not have lungs in the same sense as mammals. They have a large air sac that draws air through a rigid mass exchange apparatus. To avoid confusion with alveolar air sacs, we will call the bird's air pumping apparatus a “bellows”. The bird bellows draws (cleaned, humidified) atmospheric air through its mass exchange apparatus. The rigid mass exchange apparatus consists of channels through which air is drawn and discharged. The walls of the channels are membranes that separate the air flow from the blood flow. The exchanger has a smaller mass transfer area than a corresponding mammalian lung. However, the fresh air drawn through it has an oxygen concentration of 21%, instead of the 14% to 16% found in human air sacs. Similarly, it has almost zero percent carbon dioxide instead the 5 to 6% found in human air sacs. Table 1, enables us to compare the driving forces in a bird lung with those in a human lung. It is seen that the carbon dioxide driving force increases by a factor in excess of 4 and the oxygen driving force by up to a factor of 4.5. Furthermore, as discussed above in “Mass Transfer in respiratory aids and prosthetic lungs” the mass-transfer coefficients (U) are significantly greater. It follows from Equation 1 that, a bird can achieve an order of magnitude greater mass transfer per unit area than can a human.

The bird model has been recognized by the Applicant to provide a starting point for its solution to the prosthetic lung problem. It may even be possible to improve on the bird-lung performance by controlling the flow pattern employing fluidic logic (again requiring no moving parts). The benefits of the bellows/exchanger model are not only higher efficiency, but also greater simplicity. The order of magnitude improvement of mass transfer rate per unit area enables us to reduce the area by an order of magnitude and still support the same human metabolic rate. Thus, with only of order 10 m² equivalent surface area of prosthetic lung, a person should be as fit as a normal person with 100 m² of lung surface. Ten square meters is more readily engineered. If we are prepared to accept some deterioration from full fitness, but still better than the average smoker, we might find 5 m² surface area satisfactory.

There is also a substantial improvement in simplicity. The bellows suitable for use in the prosthetic lung herein are in essence, two elastic sacs, one for each lung. They fill the lung cavities, each being about five litres in volume. (This volume varies considerably from person to person). The bellows may be individually made, or could be manufactured in a range of standard sizes. The bellows contain no blood flow and need not be thin and fragile. They can thus be extremely robust with hope for a long maintenance-free life.

The mass exchange apparatus can be made of thin sheets of gas-permeable material. The sheets may contain a high density of parallel capillary channels through which blood flows. Alternatively, they could be two sheets closely joined with a small space between to allow blood flow. In either case, the sheets carrying the blood flow would be stacked with a small air space between each. As a further alternative, the mass exchange apparatus could be made of fine tubing (“hollow fibres”) with the air flowing around it or around the tubes. The bellows would pump the air through the spaces to create effective mass-transfer conditions. As an order of magnitude estimate, a mass exchange apparatus having a volume of 3 litres would have an air space of a litre and leave the bellows space to shift up to 2 litres of air at each breath.

The only part of the prosthetic lung that regularly moves (expands and contracts) is the bellows. This part can be made extremely robust.

The walls defining the conduits of the mass exchange apparatus are typically only a fraction of a millimetre thick. However, they will not move significantly. Thus, the exchanger will not be subject to the stresses of the alveolar air sacs, so that risk of damage is reduced. Materials of construction may be determined by gas permeability or biocompatibility considerations. Both rigid and flexible materials may be considered.

The straight air channels in the mass exchange apparatus are swept by air at significant velocity. Therefore, we may expect them to be self-cleaning.

One important design consideration is low pressure drop. The pressure drop on the blood side should be sufficiently low that the blood can be pumped through it using normal blood pressure. The design blood-side pressure drop is suitably no more than of order 1 kPa (5 inches of water, or 10 mm Hg). The design air-side pressure drop is suitably no more than 0.1 kPa (1 inch of water, 2 mm Hg). Spacing (or tube diameters) of a fraction of a millimetre (for example, 0.1 mm to 0.2 mm) allow such low pressure-drops to be achieved. The pressure drops can be achieved whilst still meeting the target total mass exchange area within a volume of order 1 litre.

Considerations in Designing an External Respiratory Aid.

The considerations in designing the mass exchange apparatus for the external respiratory aid are similar to those of the prosthetic lung. The external mass exchange apparatus(es) do not have to fit within the lung space. Hence, in principle, size is less restrictive. However, it is desirable to minimize the size for portability reasons, and it is also desirable to minimize the blood inventory outside the body. The aim is for a small, insulated, device very close to the body that will not cool the blood significantly. A larger device, with a larger external blood inventory would require the additional complication of heating and temperature control functions. Thus, the external respiratory aid should desirably not be larger than the internal prosthetic lung device. The air fed to the external respiratory aid should be cleaned, so that airborne particulates, and possible sources of infection, are minimized. The blood pump should be designed to minimize the inventory of blood. A peristaltic pump, which just squeezes the tube containing the blood, meets this requirement. Other low-volume pumps are possible. For both the blood and air side of the mass exchange apparatus, low pressure drops (similar to those of the internal device) are desirable. Low pressure drops require less power from the pumps, and hence less weight from portable power sources (batteries). There are also safety benefits. For example, employing low air pressure throughout avoids the hazard of introducing air into the blood stream, should there be a loss of integrity in the device.

It is desirable to take an approximately constant proportion of the blood flow through the external mass exchange apparatus. To this end, a simple flow controller may be employed that responds to the blood flow rate. The blood flow rate gives a measure of metabolic oxygen requirement. Thus, the controller can also adjust air flow rates and relative air/blood flow ratio to achieve desired oxygen and carbon dioxide levels. Heart rate is an approximate indicator of blood flow rate. (Depending on the person, there is a flow of approximately 100 ml per heart beat). Hence, it should be possible to control pumping rates (for both air and blood) by sensing the pulse rate.

There are benefits in extracting and returning blood from points close together in the same vein. The benefits include: (1) no vein or artery is starved of blood, (2) the blood flow through the heart and lungs is not diminished, (3) the heart is not starved of oxygen, and (4) the device can be fitted and removed with only one point of entry into a vein. In particular, benefits are achievable with one entry-point devices. Such one entry-point devices can be achieved by constructing a single flexible conduit with two flow paths in it. For example, there may be a conduit of near circular cross-section containing two flow paths each of approximately semi-circular cross-section. Alternatively, there may be two concentric tubes. In the case of two concentric tubes, the extracted blood would flow in the annulus and the returned blood in the inner tube. Suitably, an extraction head is installed at the end of a concentric tube (e.g. as shown at FIG. 4). At the point of extraction, the outer tube (annulus) may have holes, or a mesh, through which the blood is extracted. The extracted blood reverses direction to flow through the extraction tube. The returned blood flows in the same direction as the blood in the vein from which it is extracted. By suitably tapering the device at the return point, the returned flow can mingle with the residual flow in the vein with both flows at approximately the same average velocity. (The residual flow will be pulsing because it is driven by the beating heart). A concentric tube device has benefits for temperature control. Thus, if the external respiratory aid loses too much heat, the inner tube can be made heat-conducting up to the point at which it emerges from the outer tube at the mass exchange apparatus end. If heat is lost in the external apparatus, the relatively cool return flow will be heated by the counter-current flow of fresh blood in the outer tube. Thus, it is delivered to the vein at close to blood temperature. The outer flow is correspondingly cooled so that the external exchanger is at a lower temperature and loses less heat.

Suitably, the extraction point is immediately upstream of the return point. The external surface of the extraction/return head is designed so that the device can be inserted into the vein at a convenient point and then threaded to a suitable point, for example in the vena cava system. The design also allows withdrawal of the device without major surgery. In this way, the use of the external respiratory aid is easily reversible. A similar design applies for the case where the extraction and return channels are side-by-side, rather then concentric.

Referring to the section “Mass transfer in respiratory aids and prosthetic lungs”, it is seen that by adjusting both the total air flow rate and any recycle rate, independent adjustment of the blood oxygen and carbon dioxide concentrations is possible. It is anticipated that prior calibration will provide a suitable relationship between total flow rate and recycle rate. Hence, it may require only one sensor reading to control all the necessary flows.

Considerations in Designing an Intermediate Respiratory Aid

In this arrangement, one or more, mass exchange apparatus herein are fitted internally. Blood is pumped through the exchanger(s) by the patient's own circulatory system. The same area and pressure drop considerations apply as for the internal and external respiratory aids. Air is conducted into the exchanger(s) by a tube connected to an external air pump. A tube (or tubes) connected from the internal exchanger(s) conducts exhaust air outside the body for discharge to atmosphere. The air flow rate may be controlled as for the external respiratory aid. It is also possible to recycle part of the exhaust air as described for the external respiratory aid.

Desired Flow Patterns

Applicant has realized that flow patterns in the exchanger herein, and flow rates of air and blood should be arranged to provide blood oxygen/carbon dioxide relationships similar to those for natural respiration. The relevant relationships are discussed under “Mass Transfer in respiratory aids and prosthetic lungs”. The flow pattern is suitably a combination of counter-current, co-current and recycled air flow. The natural blood oxygen/carbon dioxide concentration relationships should be maintained because the body controls respiration primarily on carbon dioxide concentration in the blood. (There are also secondary controls). In order to enable the natural control mechanisms to control blood oxygen levels, the sensed carbon dioxide level must correspond to an expected oxygen level. For example, consider the case that, in normal respiration, an equilibrium partial pressure of 5.5 kPa carbon dioxide in the blood corresponds to a 12 kPa equilibrium partial pressure of oxygen in the blood. Further assume that this oxygen level is the one required in the fresh arterial blood. The apparatus herein should thus be arranged such that a 5.5 kPa carbon dioxide partial pressure corresponds to a 12 kPa oxygen level. When the body then tries to achieve 5.5% carbon dioxide partial pressure, it will actually give the desired oxygen level. The numerical values do not need to correspond exactly because, over time, the body can adjust its target carbon dioxide levels to compensate for drift in the correlation. (These compensations occur naturally when lung function slowly deteriorates. Similarly, they are established over a few days following improved lung function after transplant). However, the relationship must be monotonically decreasing. Thus, every increase in oxygen concentration must be accompanied by a decrease in carbon dioxide concentration. The precise flow pattern may be varied depending on the proportion of blood passing through the exchanger. Where the whole blood flow passes through the exchanger, the relationship from the exchanger is arranged to mimic the natural relationship. Where only a fraction of the blood is passed through the exchanger, a lower concentration of carbon dioxide from the exchanger may be appropriate, because the oxygenated blood is diluted with the remainder of the blood circulation. (In these circumstances, a higher oxygen concentration is also appropriate). Account can be taken of any residual performance of the lungs.

The apparatus herein, particularly when arranged as an external or intermediate apparatus, suitably includes a controller that senses the body's demand for oxygen. The controller would typically increase the exchange rate when more oxygen is needed, and decreases the exchange rate when less oxygen is needed. The sensing could be done for example by sensing pulse rate or breathing rate. Thus, supply of oxygen (and removal of carbon dioxide) is arranged to meet the patient's need. The patient's own natural responses indicate that need. The primary sensing organ for controlling respiration senses carbon dioxide. Hence the importance of matching the CO₂/O₂ relationship to the natural one (as described above).

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A mass exchange apparatus for use in blood/air mass exchange comprising
(a) plural blood flow conduits for defining blood flow; and
(b) plural air flow conduits for defining air flow;
wherein said plural air flow conduits and said plural blood flow conduits at least partially comprise gas-permeable membrane material, and the conduits are arranged relative to each other such as to enable transfer of oxygen from the air to the blood and transfer of carbon dioxide from the blood to the air through said membrane material.

2. A mass exchange apparatus according to claim 1, wherein said air flow is arranged to be a combination of air flow that is counter-current to the blood flow and air flow that is co-current to the blood flow.

3. A mass exchange apparatus according to claim 1, wherein the air flow includes recycled air flow.

4. A mass exchange apparatus according to claim 1, wherein the blood flow conduits and/or air flow conduits have a diameter of less than 0.5 millimetres.

5. A mass exchange apparatus according to claim 1, wherein the blood flow conduits and air flow conduits are defined by a series of plates that are separated by a distance of less than 0.5 millimetres.

6. A mass exchange apparatus according to claim 1, additionally including a sensor for sensing a patient's demand for oxygen.

7. A mass exchange apparatus according to claim 6, wherein the sensor senses the pulse rate of a patient.

8. A mass exchange apparatus according to claim 1, additionally including a controller for controlling the rate of blood/air mass exchange.

9. A mass exchange apparatus according to claim 8, wherein said controller is responsive to the sensor.

10. A mass exchange apparatus according to claim 1, wherein said gas-permeable membrane material comprises a hollow fibre membrane comprising poly-4-methylpentene-1 and having an oxygen permeation rate Q(02) at 25 C of from 1×10⁻⁶ to 3×10⁻³ (cm (STP)/cm.sec.cmHg) and an ethanol flux of from 0.1 to 100 ml/min.m, wherein said membrane has a surface comprising an ionic complex derived from: quaternary aliphatic alkylammonium salts; and heparin or a heparin derivative, and wherein said quaternary alkylammonium salts comprise a quaternary aliphatic alkylammonium salt having from 22 to 26 carbon atoms in total and a quaternary aliphatic alkylammonium salt having from 37 to 40 carbon atoms in total.

11. An association of plural mass exchange apparatus according to claim 1, defining a combined mass-transfer area of from 5 to 20 square metres.

12. A prosthetic lung comprising (a) at least one mass exchange apparatus according to claim 1; and (b) bellows or air sac means for supplying air flow to the air flow conduits.

13. A prosthetic lung according to claim 12, wherein said mass exchange apparatus locates within said bellows.

14. A respiratory aid apparatus for external connection to a patient comprising (a) at least one mass exchange apparatus according to claim 1; (b) an air pump for pumping air through said air conduits; and (c) a blood pump for pumping blood through said blood conduits.

15. A respiratory aid apparatus according to claim 14, additionally comprising an air filter for filtering the air.

16. A respiratory aid apparatus according to either claim 1, comprising two mass exchange apparatus arranged in parallel fashion.

17. A respiratory aid apparatus according to claim 1, additionally comprising a humidifier for humidifying the air.

18. A respiratory aid apparatus according to claim 1, wherein input tubing to said blood pump is arranged to provide blood extraction and return via a single entry point in a vein of a patient.

19. A respiratory aid apparatus according to claim 18, wherein said input tubing is arranged concentrically.

20. A respiratory aid apparatus according to claim 1, arranged such that extracted blood undergoes counter-current heat transfer with returned blood.

21. A respiratory aid apparatus according to claim 1, additionally comprising a heat exchanger for preheating the air.

22. An intermediate respiratory aid apparatus for internal connection to a patient comprising (a) at least one mass exchange apparatus according to claim 1; and (b) an air pump for pumping air through said air conduits.

23. An intermediate respiratory aid apparatus according to claim 22, wherein said at least one mass exchange apparatus is arranged to connect directly with a vein of a patient.
